(19) United States
(12) Patent Application Publication
(10) Pub. No.: US 2008/0175880 A1
(43) Pub. Date: Jul. 24, 2008

(54) Pyridoxal-5-Phosphate And Stent For The Treatment And Prevention Of Atherosclerosis And Restenosis

(75) Inventor: Marjorie Zettler, Winnipeg (CA)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MEIDCURE INTERNATIONAL INC., Holetown, St. James (BB)

(21) Appl. No.: 11/575,734

(22) PCT Filed: Sep. 26, 2005
(86) PCT No.: PCT/CA05/01471

§ 371 Date: Oct. 23, 2007

Related U.S. Application Data
(60) Provisional application No. 60/612,710, filed on Sep. 24, 2004.

(51) Int. Cl. A61F 2/04; A61K 31/435; A61P 9/10

(52) U.S. Cl. 424/423; 424/94.1

(57) ABSTRACT

The present invention provides method of treating and preventing vascular inflammation, atherosclerosis, restenosis, plaque destabilization and bypass graft failure comprising the administration of a therapeutically effective amount of pyridoxal-5′-phosphate or a pharmaceutically acceptable salt thereof. The present invention further provides an intravascular stent for use with a narrowed artery having at least one surface reversibly bound with pyridoxal-5′-phosphate or a pharmaceutically acceptable salt thereof.

### FIELD OF INVENTION

The present invention relates to compounds and methods for treating and preventing atherosclerosis and restenosis.

### BACKGROUND

The process of atherogenesis has long been considered to consist primarily of cholesterol build-up in the arteries. While high plasma concentrations of cholesterol (in particular, low density lipoprotein, or LDL) constitute one of the principal risk factors for the disease (1), atherogenesis is now understood to be a much more complex process. Indeed, despite widespread use of lipid-lowering agents such as stating; cardiovascular disease remains the leading cause of death in Canada, the United States, and the rest of the world (2). It has become clear that, in devising new strategies to treat this disease, it is necessary to look beyond cholesterol. Atherosclerosis is now known to be a multifactorial disease.

Percutaneous transluminal coronary angioplasty is the most commonly employed revascularization procedure for treating coronary artery disease including atherosclerosis. However, the long-term success of angioplasty is limited by the occurrence of restenosis, which is observed in 30-60% of patients within 6 months (5). Patients experiencing restenosis must often undergo repeat revascularization procedures. The introduction of stents, in particular drug-eluting stents, has reduced the incidence of restenosis to 10-30% (6) however, restenosis is still a significant problem at these rates of incidence. Compounding the problem is the fact that for patients with complex coronary artery disease, left main coronary artery disease, or infrainguinal artery disease (where the type of lesion or anatomy of the target vessel is not optimal for stenting), restenosis rates remain high (6, 7). As well, the incidence of restenosis in diabetics is approximately twice that of non-diabetics, even when stents are employed (8). Neointimal formation following angioplasty, as with atherosclerosis, is a complex process, involving inflammation, hyperhomocysteinemia, migration, proliferation, and remodeling of the vessel wall (9-11).

Present therapies fail to effectively address the multiple facets of atherosclerosis and restenosis. Presently, pharmaceutical therapies aimed at treating or preventing atherosclerosis have focused primarily on the reduction of lipids, with statins being the lipid lowering agent of choice. While statins are also potent anti-inflammatory agents, they do not address the other numerous factors (including lipidoxidation, vascular cell proliferation, hyperhomocysteinemia and thrombosis) which contribute to the development and progression of atherosclerosis. Accordingly, combination therapies have been proposed comprising one or more of lipid lowering agents, anti-thrombosis agents, and/or homocysteine lowering agents. For example, the use of statins, in combination with aspirin, beta-blockers, and angiotensin converting enzyme (ACE) inhibitors has been advocated for aggressive treatment of atherosclerosis (126).

While combination therapies comprising different classes of drugs allow for treatment of multiple risk factors, the usefulness of such therapies may be limited due to adverse drug-drug interactions. The concurrent administration of multiple drugs may result in increased toxicity and/or decreased efficacy of one or more the drugs. For example, one drug may negatively affect the metabolism of another co-administered drug. The statin, atorvastin, has been shown to reduce the effectiveness of clopidogrel, an inhibitor of platelet aggregation (127). Atorvastin, a substrate for CYP3A4, competitively inhibits CYP3A4 activation of clopidogrel.

The usefulness of combination therapies may also be limited due to poor patient compliance if the treatment regimen is overly complex, for example, if individual drugs have to be taken separately at different times of the day or if certain drugs have to taken on an empty stomach whereas the other drugs have to be taken on a full stomach. It is well established that increased treatment complexity is directly correlated with decreased patient compliance. Thus the effectiveness of a combination therapy is related not only to the pharmaceutical efficacy of the particular combination of the drugs, but also the ease and simplicity of carrying out the treatment

Pyridoxal-5′-phosphate (P5P) is a well known vitamin B6 compound. The present inventors have previously disclosed the usefulness of P5P for the treatment of cardiovascular diseases such as hypertrophy, hypertension, congestive heart failure, and ischemia (See U.S. Pat. No. 6,677,356).

Apoptosis plays a key role in regulation of the integrity of the arterial wall. During atherogenesis, deregulated apoptosis may cause abnormalities of arterial morphogenesis, wall structural stability, and metabolisms. Many biophysiologic and biochemical factors, including mechanical forces, reactive oxygen and nitrogen species, cytokines, growth factors, oxidized lipoproteins, etc. may induce or influence apoptosis of vascular cells. Apoptosis also plays a significant role in plaque destabilization. Macrophages are the most predominant cell type in atherosclerotic lesions. Recent reports have suggested that macrophage apoptosis promotes plaques destabilization in advanced atherosclerotic lesions.

Previous studies have shown that substantial amounts of adenosine triphosphate (ATP) can accumulate in the extra-cellular space under a variety of physiological and pathophysiological conditions (80-82). ATP acts on cells via P2-purinergic receptors to trigger a number of different responses including secretion, chemotaxis, proliferation, transcription factor activation and cytotoxicity (83). In addition, ATP is known to be a powerful pro-apoptotic agent mediating its effects through the specific activation of P2X7 receptors (84, 85). When ATP binds to P2X7 receptors it facilitates a rapid bi-directional flux of cations thereby triggering depolarization, collapse of the Na+ and K+ gradients, and massive influx of Ca2+. Furthermore, continued stimulation of P2X7 receptors causes formation of large, nonspecific pores, allowing permeability of molecules up to 800 Da via recruitment of a distinct pore-forming moiety (86-88). Several studies have shown that P5P inhibits the effects of extra-cellular ATP in a number of different tissue types including the heart (89, 90), vagus nerve (91), vas deferens (91) and smooth muscle cells (92).

The present inventors have previously found that P5P inhibits ATP induced calcium influx in freshly isolated adult rat cardiomyocytes (89, 93) and inhibits the positive inotropic effects of ATP on isolated perfused rat hearts (89, 90). The effects of P5P on the inhibition of apoptosis, or its ability to decrease the rate of restenosis following angioplasty, has never been contemplated and is not known.

Inflammation is a key component of the atherogenic process, from the initiation of the fatty streak to the advanced, complicated lesion (3, 94, 95). The presence of oxidized LDL and other lipids in the subendothelial space can generate an inflammatory response, causing monocytes to adhere to the vessel wall. Monocytes are transformed into macrophages, which can take up oxidized LDL to form foam cells. In addition to forming part of the fatty streak, these cells then also release cytokines and growth factors that stimulate vascular cell migration and proliferation (3, 94, 95). Platelets can also adhere to the damaged vessel wall. When activated, the platelets release their granules, which contain cytokines and growth factors that may also contribute to the migration and proliferation of vascular cells (3). Activation of platelets also leads to the formation of free arachidonic acid (3). Arachidonic acid can be converted into prostaglandins such as thromboxane. A2 (a potent vasoconstrictor and platelet aggregator) or leukotrienes (which may contribute to the inflammatory response) (3). Activated platelets which are adherent to the vessel wall also recruit other platelets, leading to the formation of a thrombus, which may cause blockage of the artery (3). Both inflammation and thrombosis are crucial components of the atherogenic process. Therefore, both must be addressed in treating the disease.

Studies have indicated that P5P levels decrease in plasma during systemic inflammatory response, such as is seen in critically or chronically ill patients (96), or those with rheumatoid arthritis (97). Similarly, plasma P5P levels are inversely correlated with C-reactive protein (a marker for inflammation) in these patients (98) as well as in patients with stroke (99). Low plasma P5P levels have also been associated with other markers for inflammation, such as tumor necrosis factor-a, in patients with rheumatoid arthritis (97). Many epidemiological studies have linked low plasma P5P levels to cardiovascular disease, leading several authors to suggest that low plasma P5P levels may be an independent cardiovascular risk factor (70, 100-104). A strong association has also been found between low plasma P5P and ischemic stroke, independent of other risk factors (105).

Interleukin-1 is an important inflammatory mediator produced in abundance by activated monocytes and macrophages (106). IL-1 biological activity is derived from two related but distinct polypeptides, IL-1α and IL-1β (106, 107). Human IL-1β is synthesized as a 31-kDa pro-cytokine that is incompetent to bind to the type 1 IL-1 receptor (108). To gain activity, pro-IL-1β must be cleaved by caspase-1 to yield a 17 kDa carboxyl terminus-derived polypeptide (109, 110). IL-1β is released from monocytes and macrophages via an atypical secretory mechanism that does not involve the endoplasmic reticulum and Golgi complex (111). Release of IL-1β from cells stimulated to produce this cytokine is generally an inefficient process. The majority of newly synthesized cytokine molecules remains cell associated and/or are degraded (112-114). To promote the efficient proteolytic cleavage of pro-IL-1β and release of the 17 kDa mature peptide, the cytokine-producing cells must be treated with a secretion stimulus such as ATP (115-117).

Extra-cellular ATP markedly accelerates the rate of processing and release of IL-1β in both monocytes and macrophages that have been primed with lipopolysaccharrides (LPS) (116-118). The ATP induced changes are mediated via the activation of P2X7 purinergic receptors (117, 119) which serve as nonselective cation channels, which facilitate the rapid influx of extra-cellular Na+ and Ca2+ and the efflux of intra-cellular K+. Prolonged or repeated stimulation of P2X7 receptors also results in the activation of nonselective pores that allow molecules ≦800 Da to diffuse into and out of the cells (120). It has been established that perturbations of cation homeostasis, specifically the loss of intra-cellular K+ and gain Na+ and Ca2+, within monocytes and macrophages result in the activation of pro-caspase-1 and thereby accelerate the processing and release of IL-1β (113, 118, 121).

Thrombosis is also implicated in the development of atherosclerois. In terms of anti-thrombotic activity, P5P has been reported to inhibit ADP, thrombin, adrenaline, platelet activating factor, and arachidonic acid-induced human platelet aggregation and 14C-5HT release in vitro (122). In this same study, thromboxane B2 generation induced by all of these agents (except arachidonic acid) was also inhibited by P5P (122). Another in vitro study indicated that P5P was able to inhibit both prostaglandin E1 and theophylline-induced platelet aggregation (123). As well, an in vivo study using plasma from patients treated with pyridoxine (100 mg twice daily for 15 days) showed that platelet aggregation induced with the agonists ADP or epinephrine was significantly inhibited, and that bleeding time was significantly prolonged (124). Finally, in a clinical study of atherosclerotic patients treated with pyridoxine, plasma antithrombin III (AT III; a potent inhibitor of the reactions of the coagulation cascade) activity was significantly increased (16). Pyridoxine treatment has also been shown to increase AT III activity in patients with genetic homocysteinuria (125).

The role of P5P treatment on inflammation, and its resulting ability decrease the rate of restenosis following angioplasty, has never been contemplated and is not known.

### SUMMARY OF INVENTION

In a first aspect, the present invention provides a method of treating or preventing a condition selected from a group consisting of: vascular inflammation, atherosclerosis, restenosis, plaque destabilization, and bypass graft failure comprising administering to a patient in need thereof a therapeutically effective amount of pyridoxal-5′-phosphate or pharmaceutically acceptable salt thereof.

In an embodiment of the invention, the method of treating vascular inflammation, atherosclerosis, restenosis, plaque destabilization, and bypass graft failure, further comprises administering a therapeutically effective amount of an anti-inflammatory agent or a cardioprotective agent.

In a second aspect, the present invention provides a method of treating atherosclerosis in a patient suffering thereof comprising the administration of a therapeutically effective amount of pyridoxal-5′-phosphate prior to the patient undergoing a percutaneous coronary intervention.

In a third aspect, the present invention provides a use of a therapeutically effective amount of pyridoxal-5′-phosphate or pharmaceutically acceptable salt thereof for the prevention or treatment of a condition selected from a group consisting of: vascular inflammation, atherosclerosis, restenosis, plaque destabilization and bypass failure.

In a fourth aspect, the present invention provides a use of pyridoxal-5′-phosphate or a pharmaceutically acceptable salt thereof for the preparation of a medicament useful for the prevention or treatment of a condition selected from a group consisting of: vascular inflammation, atherosclerosis, restenosis, plaque destabilization and bypass failure.

In a fifth aspect, the present invention provides an intravascular stent for use with a narrowed artery wherein said stent has at least one surface which is reversibly bound with pyridoxal-5′-phosphate or a pharmaceutically acceptable salt thereof.

### DETAILED DESCRIPTION

The inventors surprisingly find that P5P is effective in preventing the formation of arterial plaques in individuals at risk for developing atherosclerosis. The inventors also find that P5P is effective in slowing the progression of atherosclerosis in afflicted individuals. The inventors also find that P5P is effective for reducing the incidence and severity of restenosis in individuals having undergone a surgical intervention for the treatment of atherosclerosis.

In view of the inventors' findings, the present invention provides the use of P5P or a pharmaceutically acceptable salt thereof for treating individuals at risk for developing atherosclerosis, individuals having atherosclerosis, individuals at risk for developing restenosis and individuals having restenosis. The present invention also provides the use of P5P or a pharmaceutically acceptable salt thereof for treating and preventing vascular inflammation, plaque destabilization and bypass graft failure caused by or relating to atherosclerosis and/or restenosis. The methods of treatment according to the present invention encompass the use of P5P for modulating multiple factors which contribute to the development and progression of atherosclerosis and restenosis.

In contrast to current combination therapies, the present invention provides uses and methods of treatment which are safe, effective for ameliorating multiple risk factors, conducive to patient compliance, and low cost. The methods of treating and preventing atherosclerosis according to the invention comprise the administration of a therapeutically effective amount of P5P to a patient in need thereof. Animal and human studies show that P5P is easily tolerated and has low toxicity. P5P treatment is effective for modulating the primary factors underlying the development and progression of atherosclerosis, namely, hypercholesterolemia, lipid oxidation, vascular cell proliferation, hyperhomocysteinemia, vascular cell apoptosis, inflammation, and thrombosis. Thus, P5P provides a simple alternative to currently available combination therapies which require the administration of multiple classes of drugs which are often quite costly. As only one drug, namely P5P, needs to be administered, the ease of carrying out the methods of treatment according the invention is much greater as compared to prior art combination therapies and as such, the likelihood of patient compliance is greatly increased. In some circumstances it may be desirable to administer P5P in, conjunction with another selective anti-atherogenic agent in order to more effectively treat the patient. However, as P5P modulates multiple causative factors, fewer classes of drugs are required in order to address the equivalent number of risk factors addressed by current combination therapies. Furthermore, as P5P does not inhibit hepatic cytochrome enzymes, combination therapies comprising P5P have lower incidences of adverse drug-drug interactions.

The present inventors find that P5P advantageously modulates: (1) hypercholesterolemia and lipid oxidation, (2) vascular cell proliferation, (3) hyperhomocysteinemia, (4) vascular cell apoptosis, and (5) inflammation and thrombosis to provide substantial benefit, when administered, to individuals with or at risk of atherosclerosis and/or restenosis.

### Hypercholesterolemia and Lipid Oxidation

The present inventors confirm the lipid modulating properties of P5P. Unexpectedly, the present inventors find that the lipid modulating properties of P5P are substantially more effective than those of pyridoxine or magnesium P5P glutamate. The present inventors further find that individuals treated with P5P show clinically significant alterations to their lipid profiles as compared to the profiles of individuals treated with placebo, including decreased LDL levels, increased HDL levels, and decreased levels of oxidized lipids. Furthermore, the inventors find that the P5P-modulated changes in lipid profile are correlated with a lowered incidence of the formation of plaque lesions.

Thus, the inventors find that P5P appears to have much higher effectiveness than other vitamin B6 compounds tested, for modulating hypercholesterolemia and lipid oxidation, two known factors for atherosclerosis. The inventors also find that this modulation results in lower incidences of formation of plaque lesions, and hence would be effective in treatment of atherosclerosis and/or restenosis,

### Vascular Cell Proliferation

The present inventors find that P5P effectively inhibits vascular cell proliferation. The inventors find that P5P inhibition of vascular cell proliferation is useful in the treatment of both pre-atherosclerotic individuals and individuals having advanced atherosclerosis. The inventors also find that the anti-proliferative properties of P5P in vivo are substantially greater than those of pyridoxine. The inventors surprisingly find that P5P is effective for inhibiting vascular cell proliferation in coronary and peripheral arteries. The inventors also find that P5P is substantially more effective for inhibiting vascular cell proliferation in vivo as compared to vitamin B6. The inventors find that P5P inhibition of vascular cell proliferation can be correlated with lowered incidence of plaque formation and decreased plaque size.

Thus the inventors show that P5P inhibits vascular cell proliferation, as well as lowers incidences of formation of plaque lesions, and hence would, be effective in treatment of atherosclerosis and/or restenosis.

### Hyperhomocysteinemia

The inventors find that P5P is substantially more effective as compared to pyridoxine as a homocysteine lowering agent. Further, the inventors find that pre-atherosclerotic individuals and atherosclerotic individuals treated with P5P have clinically significant reductions in plasma homocysteine levels and lowered incidences of plaque lesions as compared to individuals treated with placebo.

Thus the inventors have shown that P5P is substantially more effective in treatment of atherosclerosis and/or restenosis.

### Vascular Cell Apoptosis

Studies conducted by the inventors find that P5P inhibition of apoptosis is related to P5P's ability to antagonize purinergic receptors, and in particular, P2X7 receptors.

The inventors find that P5P inhibits apoptosis of both cells in vascular walls and cells in plaque lesions. The inventors find that the anti-apoptosis properties of P5P appear to be related to its ability to antagonize purinergic receptors.

### Inflammation and Thrombosis

The present inventors find that P5P is an effective inhibitor of vascular inflammation and that the anti-inflammatory properties of P5P are substantially greater than the anti-inflammatory properties of pyridoxine. While the invention is not limited to any particular theory, it is believed that the effectiveness of P5P in treating and preventing atherosclerosis and restenosis is substantially related to its anti-inflammatory properties as compared to P5P's lipid lowering properties, homocysteine lowering properties and anti-thrombosis properties. The present inventors find that the anti-inflammatory properties of P5P are also related to its ability to antagonize purinergic receptors.

The present inventors find that P5P inhibits IL-1□ secretion by monocytes and macrophages which express P2X7 receptors and thereby inhibits inflammation within the vasculature. The present inventors further find that P5P is a substantially more effective purinergic receptor antagonist, and consequently anti-inflammatory agent, compared to other vitamin B6 compounds.

The present inventors find that the antithrombotic properties of P5P contribute to its antianthrogenic properties. The present inventors further find that P5P is the more effective antithrombotic agent for use in treating atherosclerosis as compared to vitamin B6.

It is to be understood that this invention is not limited to specific dosage forms, carriers, or the like, as such may vary. It is also to be understood that the terminology used herein is for the purpose of describing particular embodiments only, and is not intended to be limiting.

The present invention provides a method of treating or preventing atherosclerosis in patient comprising the administration of a therapeutically effective amount of P5P or a pharmaceutically acceptable salt thereof. The method of treatment can be used for pre-atherosclerotic individuals. Pre-atherosclerotic individuals include those which are risk of developing atherosclerosis (i.e. have one more risk factors such as increased lipids, increased homocysteine, elevated CRP levels) but have only minimal changes to the vasculature (i.e. accumulation of plaque lesions, narrowing of the arteries). In the case of pre-atherosclerotic individuals, the administration of pyridoxal-5′-phosphate is useful for preventing the onset of atherosclerosis or delaying the onset of atherosclerosis. The method of treatment can also be used with atherosclerotic individuals (i.e. those with clinically significant narrowing of the coronary or peripheral arteries) for retarding the progression of the disease by preventing further damage and by preventing destabilization of existing plaque lesions.

In another aspect, the invention provides a method of treating atherosclerosis in a patient suffering thereof comprising the administration of a therapeutically effective amount of pyridoxal-5′-phosphate or a pharmaceutically acceptable salt thereof, prior to the patient undergoing a percutaneous coronary intervention. This method is useful for atherosclerotic patients for which pharmaceutical intervention alone is insufficient. In some circumstances, the extent of the disease will be such that the only way to restore adequate blood flow is through surgical intervention such as carotid atherectomy, and in particular through a percutaneous coronary intervention.

Examples of percutaneous coronary interventions encompassed by the present invention include, but are not limited to: percutaneous transluminal coronary angioplasty (PTCA), rotational atherectomy, directional atherectomy, extraction atherectomy, laser angioplasty, implantation of intracoronary stents and other catheter devices.

In an embodiment of the invention, the patient is administered a therapeutically effective amount of P5P or pharmaceutically acceptable salt thereof, for between 1 and 30 days prior to undergoing the percutaneous coronary intervention and more preferably, 14 days prior to the intervention. In a further embodiment of the invention, the method comprises the further step of administering a therapeutically effective amount of P5P or a pharmaceutically acceptable salt thereof, following the percutaneous coronary intervention. The duration of post-operative treatment with P5P will depend on a particular patient's need. In some circumstances, long term treatment and even indefinite treatment may be desirable. In other circumstances, short term treatment may be desirable. In a preferred embodiment, P5P or a pharmaceutically acceptable salt thereof is administered between 1 and 30 days following the percutaneous intervention. In a further preferred embodiment, P5P or a pharmaceutically acceptable salt thereof is administered for at least 30 days following the percutaneous intervention.

In some circumstances, it may be desirable to co-administer an additional cardioprotective agent following the percutaneous coronary intervention. Examples of cardioprotective agents which may be administered with P5P or its pharmaceutically acceptable salt include platelet aggregation inhibitors such as: thromboxane A2 inhibitors (e.g. acetylsalicylic acid (ASA)), glycoprotein IIb/IIIa inhibitors (e.g. abciximab, eptifibatide, tirofiban, lamifiban, xemilofiban, orbofiban, sibrafiban; fradafiban, roxifiban, lotrafiban), adenosine phosphate inhibitors (e.g., clopidogrel, dipyridamole, sulfinpyrazone), fibrinogen-platelet binding inhibitors (ticlopidine), or a platelet c-AMP phosphodiesterase inhibitors, such as dipyridamole or cilostazol, or pentoxifylline (trental).

In another aspect, the present invention provides a method of treating or preventing vascular inflammation in a patient comprising administering a therapeutically effective amount of a pyridoxal-5′-phosphate or pharmaceutically acceptable salt thereof. The patient to be treated may be an individual which is pre-atherosclerotic, and which case, the administration of P5P is particularly useful for not only reducing vascular inflammation but also in reducing the likelihood of developing atherosclerosis.

The present invention further provides a method of treating or preventing restenosis in a patient suffering thereof or at risk thereof, comprising the administration of a therapeutically effective amount of a pyridoxal-5′-phosphate or pharmaceutically acceptable salt thereof.

The restenosis treated by the methods according to the invention may be the result of a surgical intervention, and in some circumstances a percutaneous coronary intervention, examples of which are discussed above.

In another aspect, the invention provides a method of treating or preventing bypass graft failure comprising administering to a person in need thereof, a therapeutically effective amount of pyridoxal-5′-phosphate or a pharmaceutically acceptable salt thereof. In one embodiment of the invention, the bypass graft failure may be an artery graft failure, and preferably a coronary artery graft failure. In another embodiment of the invention, the bypass graft failure is a vein graft failure. The vein graft failure may be a peripheral or coronary vein graft failure.

The present invention further provides a method of treating atherosclerosis or restenosis comprising administering to a person in need thereof, a therapeutically effective amount of: (a) pyridoxal-5′-phosphate or a pharmaceutically acceptable salt thereof and (b) an anti-inflammatory agent.

Anti-inflammatory agents which can be used to practice the invention include but are not limited to: COX-2 inhibitors such as celecoxib, rofecoxib, valdecoxib; pyralones such as phenylbutazone; fenamates such as mefanamic acid, meclofenamate; salicylic acid derivative such as diflunisal; acetic acid derivatives such as diclofenac, indomethacin, sulindac, etodolac, ketorolac, nabumetone, tolemetin; propionic acid derivatives such as ibuprofen, fenoprofen, fluribiprofen, carprofen, ketoprofen, naproxen; tiaprofenic acid, oxaprozin; oxicams such as piroxicam, tenoxicam, meloxicam; biological response modifiers such as anakinra, etanercept, infliximab; corticosteriods such as betamethasone, cortisone, dexamethasone, prednisolone, methylprednisolone, prednisone, triamcinolone; cytotoxics such as azathioprine, methotrextate; gold preparations such as aurothioglucose, aurothiomalate, auranofin; hydroxychloroquine; sulfasalazine D-penicillamine; minocycline; azathioprine; cyclosporine; or lefunomide.

The methods of treatment encompassed by the present invention can be employed with mammalian patients, and more preferably human patients. The methods of treatment encompassed by the present invention may also be employed with diabetic patients.

The methods of treatment according to the invention comprise the administration of a therapeutically effective amount of pyridoxal-5′-phosphate or a pharmaceutically acceptable salt thereof. By an “effective amount” or a “therapeutically effective amount” of a pharmacologically active agent is meant a nontoxic but sufficient amount of the drug or agent to provide the desired effect. In a combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The therapeutic effective amount of any of the active agents encompassed by the invention will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastro-intestinal tract, the hepatobillary system and the renal system. In combination therapies, it may be desirable or effective to give a lower amount of one or more of the compounds administered. Methods for determining dosage and toxicity are well known in the art with studies generally beginning in animals and then in humans if no significant animal toxicity is observed. The appropriateness of the dosage can be assessed by monitoring: blood pressure, lipid levels, CRP levels, and homocysteine levels. Where the dose does not improve metabolic, vascular and/or endothelial function or reduce blood pressure following at least 2 to 4 weeks of treatment, the dose can be increased.

Generally, the therapeutically effective amount of P5P is between 0.1 and 100 mg/kg of body weight per day. In an embodiment of the invention, the preferred therapeutically effective amount of P5P is between 0.5 and 50 mg/body weight per day. In a further embodiment of the invention, the preferred therapeutically effective amount of P5P is between 1 and 25 mg/kg body weight per day. In yet another embodiment of the invention, the therapeutically effective amount of P5P is preferably between 1 and 15 mg/kg body weight per day.

P5P or its pharmaceutically acceptable salt may be administered to patient in need thereof by any suitable route. Preferably, the methods of treatment of the according to the invention comprise the oral administration of P5P or a pharmaceutically salt thereof. Preferred oral dosage forms contain a therapeutically effective unit dose suitable for a once-daily oral administration. Typically, the unit dosage for P5P will be between 100, 300, 750 and 1000 mg/day.

In some instances, it may be preferably to administer P5P or its pharmaceutically salt thereof in situ at a specific site of vascular damage. The present invention provides an intravascular stent for in situ delivery of P5P or its pharmaceutically acceptable salt thereof. The intravascular stent according to the invention is for use within a narrowed artery and has at least one surface which is reversibly bound with pyridoxal-5′-phosphate or a pharmaceutically acceptable salt thereof. In a preferred embodiment, the exterior of the stent may be coated with a physiologically compatible matrix adapted for time delayed release of the reversibly bound pyridoxal-5′-phosphate or pharmaceutically acceptable salt thereof upon implantation of the stent within the artery to be treated.

In one embodiment, the intravascular stent is prepared using between 10 mg and 10,000 mg of pyridoxal-5′-phosphate or a pharmaceutically acceptable salt thereof. In another embodiment, the intravascular stent is prepared using between 1000 mg and 10,000 mg of pyridoxal-5′-phosphate or a pharmaceutically acceptable salt thereof. In further embodiment, the intravascular stent is prepared using between 1000 mg and 7,500 mg of pyridoxal-5′-phosphate or a pharmaceutically acceptable salt thereof. In a still further embodiment, the intravascular stent is prepared using between 1000 mg and 5000 mg of pyridoxal-5′-phosphate or a pharmaceutically acceptable salt thereof. In yet a further embodiment, the intravascular stent is prepared using between 10 mg and 1000 mg of pyridoxal-5′-phosphate or a pharmaceutically acceptable salt thereof.

The intravascular stent according to the invention may be prepared using appropriate prior art methods and materials. The construction of intravascular stents for use with narrowed arteries is well known in the art. The preparation of intravascular stents adapted for in situ drug delivery is also well known in the art.

Although the present invention has been described with reference to illustrative embodiments, it is to be understood that the invention is not limited to these precise embodiments, and that various changes and modifications may be effected therein by one skilled in the art. All such changes and modifications are intention to be encompassed in the appended claims.

### EXAMPLE ONE

### Pyridoxal 5′-phosphate Inhibits Pro-Apoptotic Effects of ATP on Cells Expressing P2X7 Receptors

Cell Lines—HEK 293 cell line (human), is stabily transfected with the P2X7 receptor [9] to produce HEK 293 P2X7 cells. This cell line does not express any of the other P2X receptors; however it does express P2Y1 and P2Y2 receptors [15].

Cell Viability—HEK 293 P2X7 cells are treated with ATP and/or BzATP to induce cell death (Wen et al (2003) Mol Pharmacol-63:706-713). Cell viability is determined using an MTT assay (Wen et al., 2003). Various doses of P5P are added to the cells to determine its ability to inhibit ATP induced cell death. OxATP (Murgia M et al (1993) J Biol Chem 268:8199-8203), PPADS (Chessell I P et al (1998) Br J Pharmacol 124:1314-1320) and/or KN-62 (Humphreys B D et al (1998) Mol Pharmacol 54:22-32) are used as positive controls.

Apoptosis—HEK-293 P2X7 (human) cells are treated with ATP and/or BzATP to induce apoptosis (Wen et al., 2003). The level of apoptosis is determined using a Cell Death Detection ELISA, which is based on DNA laddering. Various doses of P5P are added to the cells to determine its ability to inhibit ATP induced apoptosis. OxATP (Murgia et al., 1993), PPADS (Chessell I P et al., 1998) and/or KN-62 (Humphreys B D et al., 1998) are used as positive controls.

Membrane Blebbing/Pore Formation—HEK 293 P2X7 (human) cells are treated with ATP and/or BzATP to induce membrane blebbing or pore formation (Virginio C et al (1999) J Physiol 519:335-346. Verhoef P A et al (2003) J Immunol 170:5728-5738). Membrane blebbing is assessed using YO-PRO 1 (Virginio C et al., 1999. Verhoef P A et al., 2003). Various doses of P5P are added to the cells to determine its ability to inhibit ATP induced membrane blebbing. OxATP (Murgia et al., 1993), PPADS (Chessell I P et al., 1998) and/or KN-62 (Humphreys B D et al., 1998) are used as positive controls.

Control—All the experiments are carried out in wild type HEK 293 cells as a control.

Results—ATP and BzATP do not induce cell apoptosis in wild type HEK 292 cells. P5P treated HEK 293 P2X7 cells resist ATP or BzATP mediated cell apoptosis as compared to untreated HEK 293 P2X7 cells. The results show that ATP and BzATP induce cell apoptosis in P2X7 expressing cells. The results further show that P5P inhibits ATP mediated cell apoptosis in P2X7 expressing cells.

### EXAMPLE TWO

### Pyridoxal 5′-phosphate Inhibits ATP Mediated IL-1β Secretion by Monocytes and Macrophages Expressing P2X7 Receptors

LPS primed THP-1 cells are treated with ATP to induced IL-1β secretion (Grahames C B A et-al (1999) Br J Pharm 127:1915-1921). IL-1β secretion levels are determined by ELISA. Various doses of P5P are added to the cells to determine its ability to Inhibit the ATP induced IL-1β secretion. OxATP (Grahames et al., 1999), PPADS (Grahames et al., 1999), and/or KN-62 (Grahames et al., 1999) are used as positive controls.

Results—ATP treatment of LPS primed THP-1 cells induces IL-1□□secretion. Treatment of LPS primed THP-1 cells with purinergic receptor antagonist OxATP, PPADS or KN-62 inhibits IL-1β secretion. P5P treatment of LPS primed THP-1 cells inhibits IL-1β secretion.

### EXAMPLE THREE

### Pyridoxal 5′-phosphate Ameliorates Restenosis in Rabbits Suffering Aortic Balloon Injury

Rabbit Aortic Balloon-Injury Model—Forty male New Zealand White rabbits (2.5-3.0 kg) are used. Rabbits are fed regular rabbit chow (control, 20 rabbits) or a 1% cholesterol diet (treated, 20 rabbits) for 8 weeks. Half of each group receives 10 mg/kg P5P (provided by CanAm Bioresearch Inc.) daily by gavage, with the other half receiving saline by gavage. After 4 weeks, a 3F Fogarty embolectomy catheter (Baxter) is inserted into the right femoral artery of each rabbit, advanced 25 cm proximally, and then withdrawn to the origin with the balloon inflated to 0.2 mL saline, a step repeated twice (Lau et al., Probucol promotes functional reendothelialization in balloon-injured rabbit aortas. Circulation 2003; 107(15):2031-6). At 8 weeks, the right and left femoral arteries are harvested from each animal (left femoral arteries serve as uninjured controls). Segments for histology are pressure-perfused with formalin, stored in. 70% (vol/vol) ethanol, and then embedded in paraffin.

Histology—One 5 μm thick cross-section is taken from each of 6 segments of the femoral aorta and stained with hematoxylin and eosin. Morphometric analysis of the 6 arterial cross-sections per animal is performed using Imagespace software (Molecular Dynamics). The intimal and medial areas of each arterial cross-section specimen is measured, and the average intimal/medial area ratio is determined for each group.

Inflammatory Markers, Lipid Profile, and Homocysteine Levels—At the beginning and end of the study, and every 2 weeks throughout, blood samples are collected through a catheter inserted in the ear artery of conscious rabbits. Plasma interleukin-6 (IL-6) levels are measured with quantitative sandwich enzyme immunoassay employing commercial kits for rats (R&D Systems) (Oubina et al., Synergistic effect of angiotensin-converting enzyme (ACE) and 3-hydroxy-3-methylglutaryl-CoA (HMG-CoA) reductase inhibition on inflammatory markers in atherosclerotic rabbits. Clin Sci (Lond). 2003; 105(6):655-62). CRP plasma levels are measured with an immunoassay kit for rabbits (Alpco Diagnostics). Plasma cholesterol levels are measured using colorimetric reactions employing commercial kits (Roche Diagnostics). Homocysteine levels are determined by high performance liquid chromatography, as previously described (Zhloba and Blashko, Liquid chromatographic determination of total homocysteine in blood plasma with photometric detection. J Chromatogr B Analyt Technol Biomed Life Sci. 2004; 800(1-2):275-80).

Evaluation of Endothelial Function by Ultrasound—Ultrasound evaluation of endothelial function of the femoral aorta is performed at the beginning and end of the study, and every 2 weeks throughout (Drolet et al., Early endothelial dysfunction in cholesterol-fed rabbits: a non-invasive in vivo ultrasound study. Cardiovasc Ultrasound. 2004; 2(1):10). Rabbits are sedated using midazolam (0.5 mg/kg), butorphanol (0.5 mg/kg) and ketamine (30 mg/kg) IM. Marginal ear vein and artery are cannulated for drug infusions and arterial blood pressure monitoring, respectively. Heart rate is monitored continuously throughout the procedure. The femoral aorta is located using two-dimensional and color Doppler ultrasound. Image settings are optimized for definition of the endothelial-blood interface. All studies are performed with a vascular 7.5 MHz probe coupled to a Sonos 5500 echograph (Phillips).

Once the imaging of the aorta is optimal, the animals receive the following drug perfusions I.V. sequentially for 2 minutes each: 1) saline at 1 ml/min; 2) acetylcholine (Ach) at 0.05 μg/ml/min and Ach at 0.5 μg/ml/min. Nitroglycerin (5 μg/ml/min) is used as positive control. At the end of a drug infusion, blood pressure is allowed to come back to baseline for at least one minute before the next infusion is started. Images of the femoral aorta are recorded continuously through the entire procedure on standard S-VHS videotape for off-line analysis.

Statistical Analyses—Results are analyzed by 1-way ANOVA followed by application of the Turkey test to assess the significance of specific intergroup differences. Probability values of p<0.05 are regarded as significant.

Results—P5P treatment correlates with improved lipid profile, lowered homocysteine levels, lowered CRP levels and improved endothelial function. P5P treatment correlates with decreased incidence of restenosis following PCI.

### EXAMPLE FOUR

### Pyridoxal 5′-phosphate Ameliorates Restenosis in Angioplasty Patients

Study Design and Population—The study is a multicenter, double-blind, placebo-controlled, randomized trial. The protocol is approved by institutional review boards prior to commencement. Patients referred for elective percutaneous coronary interventional (PCI) are evaluated 14 days before their scheduled procedures. Eligible patients are asked to provide written informed consent and undergo medical history, physical examination, electrocardiography, hematology, and clinical biochemistry. Patients are eligible if they are scheduled to undergo PCI with or without stenting on one native coronary artery and have one de novo target lesion with luminal narrowing 50%. Subjects who have severe liver disease or serum creatinine 200 μmol/L; myocardial infarction within the past 7 days; left main stenosis >50%; ejection fraction <30%; have had PCI for another lesion in the preceding 6 months; are being treated for a restenotic lesion; or have scheduled atherectomy, brachytherapy, or PCI of a bypass graft before randomization are excluded. A total of 120 patients are enrolled, with 60 randomized to receive P5P and 60 to receive placebo.

The primary endpoint is clinical restenosis requiring target lesion revascularization at six months. The secondary end points are major adverse cardiac events (MACE) including death, myocardial infarction and revascularization.

Randomization and Drug Regimen—Patients are randomly assigned to receive P5P (provided by CanAm Bioresearch Inc.) 750 mg once day or placebo beginning 14 days before scheduled PCI. P5P or matched placebo is administered as 3 tablets given once daily. All patients will also receive an extra dose of P5P or matched placebos on the evening before PCI, according to random treatment assignment. After PCI, all patients are maintained on their assigned study regimen for 4 additional weeks.

PCI and Follow-Up Evaluation—PCI with or without stent placement and post-PCI management will be performed according to current clinical practice. ECGs are obtained before PCI, immediately thereafter, and the morning after PCI. Creatine kinase, creatine kinase-MB fraction, and troponin I are measured on the evening after PCI and the following morning. Patients are discharged after PCI with 4 weeks of the study medication. Aspirin 325 mg/d is given from the time of recruitment and for the entire study duration. All-patients treated with stents also receive clopidogrel 75 mg/d for 30 days after PCI. Patients will return at 1 Month, 3 months and 6 months for clinical evaluation and drug accountability. Patients are assessed for ischemic symptoms and adverse events, whether or not they were related to the study medication or PCI procedure. Blood chemistry values assessed at baseline are measured again at PCI discharge and at follow-up visits. Patients are readmitted for follow-up catheterization and IVUS 5 to 7 months after PCI. Those in whom catheterization is performed for clinical reasons before the fifth month return for repeat IVUS examination at 5 to 7 months if no definite restenosis is present on one dilated site.

IVUS Examinations and Measurements—IVUS examinations are performed with 30-MHz, 3.2 F ultrasound catheters (Tardif et al., Canadian Antioxidant Restenosis Trial (CART-1) Investigators. Effects of AGI-1067 and probucol after percutaneous coronary interventions. Circulation. 2003; 107(4):552-8). IVUS studies are recorded before, PCI whenever possible and are always performed after PCI (after final balloon inflation) and at follow-up (before any subsequent intervention). IVUS is always preceded by an intracoronary injection of nitroglycerin (0.3 mg). All IVUS images are interpreted by experienced technicians supervised by a cardiologist, all of them blinded to treatment assignment. The preintervention, post-PCI, and follow-up studies are analyzed side by side.

Quantitative Coronary Angiography—Control angiography after PCI and at follow-up are preceded by intracoronary administration of nitroglycerin (0.3 mg). Quantitative analysis is performed to determine dichotomous restenosis rates, A PCI segment is defined as restenotic if diameter stenosis is 50% at follow-up, with an increase of 15% in the degree of stenosis compared with the post-PCI angiogram.

Inflammatory Markers, Lipid Profile, Homocysteine Levels and Endothelial Function—Blood samples of inflammatory markers, biochemistry, and lipid profile are drawn at hospital admission and again at 1 month, 3 months and 6 months. CRP levels are determined by nephelometry, using a high-sensitivity assay (Dade Behring Marburg GmbH), and IL-6 levels will be evaluated by enzyme-linked immunosorbent assay (R&D Systems) (Monakier et al., Rofecoxib, a COX-2 inhibitor, lowers C-reactive protein and interleukin-6 levels in patients with acute coronary syndromes. Chest. 2004; 125(5):1610-5). Homocysteine levels are determined by high performance liquid chromatography, as previously described (Zhloba and Blashko, Liquid chromatographic determination of total homocysteine in blood plasma with photometric detection. J Chromatogr B Analyt Technol Biomed Life Sci. 2004; 800(1-2):275-80). Endothelial function is evaluated noninvasively using high-frequency ultrasound of the brachial artery, assessing blood flow response to hyperemia (endothelium-dependent vasodilatation) and nitroglycerin spray (endothelium-independent vasodilatation) (Dupuis et al., Cholesterol reduction rapidly improves endothelial function after acute coronary syndromes. The RECIFE (reduction of cholesterol in ischemia and function of the endothelium) trial. Circulation. 1999; 99(25):3227-33).

Statistical Analysis—IVUS and other continuous end points are analyzed with a 2-way ANOVA. Statistical analysis of CRP, IL-6 levels, lipid profile, homocysteine levels, and endothelial function are performed by the Wilcoxon test, comparing the 1-month, 3-month and 6 month results to the baseline values of each group. A probability value <0.05 is considered statistically significant.

Results—P5P treatment correlates with improved lipid profile, lowered homocysteine levels, lowered CRP levels and improved endothelial function. P5P treatment correlates with decreased incidence of restenosis following PCI.

### REFERENCES

1. Steinberg D. Low density lipoprotein oxidation and its pathobiological significance. J Biol Chem 1997; 272:20963-20966.
2. Braunwald E. Shattuck Lecture—Cardiovascular medicine at the turn of the millennium: triumphs, concerns, and opportunities. N Engl J Med 1997; 337:360-369.
3. Ross R. Atherosclerosis—an inflammatory disease. N Engl J Med 1999; 340(2):115-126.
4. Libby P. Vascular biology of atherosclerosis: overview and state of the art. Am J Cardiol 2003; 91(suppl):3A-6A.
5. Bauters C, Isner J M. The biology of restenosis. In: Topol E J, ed. Textbook of Cardiovascular Medicine. Philadelphia, Pa.: Lippincott-Raven; 1998:2465-2490.
6. Woods T C, Marks A R. Drug-eluting stents. Annu Rev Med. 2004; 55:169-78.
7. Sayers R D, Jones L, Varty K, Allen K, Morgan J D, Bell P R, London N J. The histopathology of infrainguinal vein graft stenoses. Eur J Vasc Surg. 1993; 7(1):16-20.
8. Suselbeck T, Latsch A, Siri H, Gonska B, Poerner T, Pfleger S, Schumacher B, Borggrefe M, Haase K K. Role of vessel size as a predictor for the occurrence of in-stent restenosis in patients with diabetes mellitus. Am J Cardiol. 2001; 88(3):243-7.
9. Welt F G, Rogers C. Inflammation and restenosis in the stent era. Arterioscler Thromb Vasc Biol 2002; 22:1769-76.
10. Ross R. Atherosclerosis—an inflammatory disease. N Engl J Med 1999; 340:115-26.
11. Schnyder G, Roffi M, Flammer Y, Pin R, Hess O M. Association of plasma homocysteine with restenosis after percutaneous coronary angioplasty. Eur Heart J 2002; 23:726-733.
12. Kandzari D E, Labinaz M, Cantor W J, Madan M, Gallup D S, Hasselblad V, Joseph D, Allen A, Green C, Hidinger K G, Krucoff M W, Christenson R H, Harrington R A, Tcheng J E. Reduction of myocardial ischemic injury following coronary intervention (the MC-1 to Eliminate Necrosis and Damage trial). Am J Cardiol 2003; 92(6):660-664.
13. Palinski W, Rosenfeld M E, Yla Herttuala S, Gurtner G C, Socher S S, Butler S W, Parthasarathy S, Carew T E, Steinberg D, Witztum J L. Low density lipoprotein undergoes oxidative modification in vivo. Proc Natl Acad Sci USA 1989; 86:1372-1376.
14. Berliner J A, Heinecke J W. The role of oxidized lipoproteins in atherogenesis. Free Radic Biol Med 1996; 20:707-727.
15. Rinehart J F, Greenberg L D. Vitamin B6 deficiency in the rhesus monkey with particular reference to the occurrence of atherosclerosis, dental caries, and hepatic cirrhosis. Am J Clin Nutr 1956; 4:318-328.
16. Mushett C W, Emerson G A. Arteriosclerosis in pyridoxine-deficient monkeys and dogs. Fed Proc 1956; 15:526.
17. Kuzaya F. Experiment on arteriosclerosis and arteriolosclerosis induced in pyridoxine deficient monkeys and their recovery. J Primatol 1962; 3:77.
18. Greenberg L D, Rinehart J F. Plasma cholesterol levels of cholesterol fed control and pyridoxine deficient monkeys. Proc Soc Exper Biol Med 1951; 76:580-583.
19. Kleinsorge von H, Zielke A. Untersuchung uber die Einwirkung verschiedener ungesattigter Fettsauren und der Vitamine B6 and E auf die tierexperimentalle Arteriosklerose und das Verhalten der Serumlipoide beim Menschen. Therap Umsch 1959; 16: 367-380.
20. Kuzuya F. Reversibility of atherosclerosis in pyridoxine-deficient monkeys. Atherosclerosis IV, Proceedings of the 4^{th} International Symposium. Berlin: Springer-Verlag, 1977:275-280.
21. Vijayammal P L, Kurup P A. Pyridoxine and atherosclerosis, Role of pyridoxine in the metabolism of lipids and glycocamines in rats fed normal and high fat, high cholesterol diets containing 16% casein. Aust J Biol Sci 1978; 31:7-20.
22. Sheikh-Ali S D. The Influence of vitamin B6 on the blood lipids and the cholesterolaemia under clinical and experimental conditions. Ter Arkh 1959; 9:90-93
23. Brattstrom L, Stavenow L, Galvard H, Nilsson-Ehle P. Berntorp E, Jerntorp P, Elmstahl S, Pessah-Rasmussen H. Pyridoxine reduces cholesterol and low density lipoprotein and increases antithrombin III activity in 80-year-old men with low plasma pyridoxal 5-phosphate. Scand J Clin Lab Invest 1990; 50:873-877.
24. Frolova I A, Oleneva V A, Sirota I I, Nikiforova G D. Effect of pyridoxine on patterns of lipid metabolism in patients with alimentary obesity. Vopr Med Khim 1975; 21:299-306.
25. Arnadottir M, Brattstrom L, Simonsen O, Thysell H, Hultberg B, Andersson A, Nilsson-Ehle P. The effect of high-dose pyridoxine and folic acid supplementation on serum lipid and plasma homocysteine concentrations in dialysis patients. Clin Nephrol 1993; 40(4):236-240.
26. De Gomez Dumm N T, Giammona A M, Touceda L A. Variations in the lipid profile of patients with chronic renal failure treated with pyridoxine. Lipids Health Dis 2003; 2(1):7.
27. Jain S K, Lim G. Pyridoxine and pyridoxamine inhibits superoxide radicals and prevents lipid peroxidation, protein glycosylation, and (Na⁺+K⁺)-ATPase activity reduction in high glucose-treated human erythrocytes. Free Radic Biol 2001; 30:232-237.
28. Ravichandran V, Selvam R. Increased lipid peroxidation by vitamin B6 deficient rats. Biochem Int 1990; 21:599-605.
29. Nakamura N, Ishikawa M. Effect of vitamin B6 group in cholesterol and fatty acid biosynthesis. Hokkaido Igaku Zasshi 1975; 50(4):403-409.
30. Yamada K, Fujii T, Motoyasu M. Influence of pyridoxal phosphate upon arteriosclerosis. Vitamins 1961; 22:136-137.
31. Weiss N, Feussner A, Hailer S, Spengel F A, Keller C, Wolfram G. Influence of folic acid, pyridoxal phosphate and cobalamin on plasma homocyst(e)ine levels and the susceptibility of low-density lipoprotein to ex-vivo oxidation. Eur J Med Res 1999′; 4(10):425-432.
32. Selmer D, Senekowitsch-Schmidtke R, Schneider W, Elstner E F. Binding and uptake of 125 iodine-labelled, oxidized low density lipoprotein by macro phages: comparison of the effects of alpha-tocopherol, probucol, pyridoxal-5′-phosphate and magnesium-pyridoxal-5′-phosphate-glutamate. Z Naturforsch 1997; 52c:97-104.
33. Panagiotopoulos T, Ketelsen U P, Schmidt A, Heuck C C. Long-term effect of magnesium pyridoxal 5-phosphate glutamate in rabbits developing hypercholesterolemia. Arzneimittelforschung. 1986; 36(8):1210-1215.
34. Khayyal M T, Khayyal M A, Sharaf H M, el-Sherbeeny M, Okpanyi S N, Schneider W. Effect of magnesium pyridoxal 5-phosphate glutamate on vascular reactivity in experimental hypercholesterolemia. Drugs Exp Clin Res. 1998; 24(1):29-40.
35. Kirsten R, Heintz B, Nelson K, Sieberth H G, Oremek G, Hasford J, Speck U. Magnesium pyridoxal 5-phosphate glutamate reduces hyperlipidaemia in patients with chronic renal insufficiency. Eur J Clin Pharmacol. 1988; 34(2):133-137.
36. Schuitemaker G E, van der Pol G A, Aretz C P, Dinant G J. A placebo-controlled, double-blind, randomised trial of magnesium-pyridoxal-5′-phosphate-glutamate for hypercholesterolaemia and other clinical-chemical risk factors of cardiovascular disease in a primary care setting. Eur J Clin Pharmacol. 2001 March; 56(12):857-863.
37. Kogl C, Schneider W, Elstner E F. The influence of magnesium-pyridoxal-5′-phosphate-glutamate in comparison with probucol, alpha-tocopherol and trolox on copper-induced oxidation of human low density lipoprotein in vitro. Biochem Pharmacol. 1994 Jun. 15; 47(12):2201-2206.
38, Zettler M E, Pierce G N. Growth-promoting effects of oxidized low density lipoprotein. Can J Cardiol 2001; 17(1):73-79.
39. Schwartz S M, Campbell G R, Campbell J H. Replication of smooth muscle cells in vascular disease. Circ Res 1986; 58:427-444.
40. Tryfiates G P, Morris H P, Sonidis G P. Vitamin B6 and cancer (review). Anticancer Res 1981; 1(5):263-267.
41. Komatsu S, Yanaka N, Matsubara K, Kato N. Antitumor effect of vitamin B6 and its mechanisms. Biochim Biophys Acta 2003; 1647(1-2):127-30.
42. DiSorbo D M, Litwack G. Vitamin B6 kills hepatoma cells in culture. Nutr Cancer 1982; 3(4):216-222.
43. Shultz T D, Santamaria A G, Gridley D S, Stickney D R, Slater J M, Effect of pyridoxine and pyridoxal on the in vitro growth of human malignant melanoma. Anticancer Res 1988; 8:1313-1318.
44. DiSorbo D M, Wagner R, Nathanson L. In vivo and in vitro inhibition of B16 melanoma growth by vitamin B6. Nutr Cancer 1985; 7(1-2):43-52.
45. Gridley D S, Stickney D R, Nutter R L, Slater J M, Shultz T D. Suppression of tumor growth and enhancement of immune status with high levels of dietary vitamin B6 in BALB/c mice. J Natl Cancer Inst 1987; 78(5):951-959.
46. Komatsu S I, Watanabe H, Oka T, Tsuge H, Nii H, Kato N. Vitamin B-6-supplemented diets compared with a low vitamin B-6 diet suppress azoxymethane-induced colon tumorigenesis in mice by reducing cell proliferation. J Nutr 2001; 131(8):2204-2207.
47. Komatsu S I, Watanabe H, Oka T, Tsuge H, Kato N. Dietary vitamin B6 suppresses colon tumorigenesis, 8-hydroxyguanosine, 4-hydroxynonenal, and inducible nitric oxide synthase protein in azoxymethane-treated mice. J Nutr Sci Vitaminol (Tokyo) 2002; 48(1):65-68.
48. Slattery M L, Potter J D, Coates A, Ma K N, Berry T D, Duncan D M, Caan B J. Plant foods and colon cancer: an assessment of specific foods and their related nutrients (United States). Cancer Causes Control 1997; 8(4):575-590.
49. Jansen M C, Bueno-de-Mesquita H B, Buzina R, Fidanza F, Menotti A, Blackburn H, Nissinen A M, Kok F J, Kromhout D. Dietary fiber and plant foods in relation to colorectal cancer mortality: the Seven Countries Study. Int J Cancer 1999; 81(2):174-179.
50. Hartman T J, Woodson K, Stolzenberg-Solomon R, Virtamo J, Selhub J, Barrett M J, Albanes D. Association of the B-vitamins pyridoxal 5′-phosphate (B(6)), B(12), and folate with lung cancer risk in older men. Am J Epidemiol 2001; 153(7):688-694.
51. Key T J, Silcocks P B, Davey G K, Appleby P N, Bishop D T. A case-control study of diet and prostate cancer. Br J Cancer 1997; 76(5):678-687.
52. Martial J, Zaldivar J, Bull P, Venegas A, Valenzuela P. Inactivation of rat liver RNA polymerases I and II and yeast RNA polymerase I by pyridoxal 5′-phosphate. Evidence for the participation of lysyl residues at the active site. Biochemistry 1975; 14(22):4907-4911.
53. Venegas A, Martial J, Valenzuela P. Active site-directed inhibition of E. coli DNA-dependent RNA polymerase by pyridoxal 5′-phosphate. Biochem Biophys Res Commun 1973; 55(4):1053-1059.
54. Modak M J. Observations on the pyridoxal 5′-phosphate inhibition of DNA polymerases. Biochemistry 1976; 15(16): 3620-3626.
55. Diffley J F. Affinity labeling the DNA polymerase alpha complex. I. Pyridoxal 5'-phosphate inhibition of DNA polymerase and DNA primase activities of the DNA polymerase alpha complex from Drosophila melanogaster embryos. 3 Biol Chem 1988; 263(29):14669-14677.
56. Matsubara K, Komatsu S, Oka T, Kato N. Vitamin B6-mediated suppression of colon tumorigenesis, cell proliferation, and angiogenesis (review). J Nutr Biochem 2003; 14(5):246-250.
57. Kopelovich L, Wolfe G. Modification of ribonucleic acid by vitamin B6. 1. Specific interaction of pyridoxal 5′-phosphate with transfer ribonucleic acid. Biochemistry 1977; 16(16):3721-3726.
58. Srivastava S K, Beutler E. The effect of normal red cell constituents on the activities of red cell enzymes. Arch Biochem Biophys 1972; 148(1):249-255.
59. Basu A, Tirumalai R S, Modak M J. Substrate binding in human immunodeficiency virus reverse transcriptase. An analysis of pyridoxal 5′-phosphate sensitivity and identification of lysine 263 in the substrate-binding domain. J Biol Chem 1989; 264(15):8746-8752.
60. Matsubara K, Ma sumoto H, Mizushina Y, Lee J S, Kato N. Inhibitory effect of pyridoxal 5′-phosphate on endothelial cell proliferation, replicative DNA polymerase and DNA topoisomerase. Int J Mol Med 2003; 12(1):51-55.
61. Matsubara K, Mori M, Matsuura Y, Kato N. Pyridoxal 5′-phosphate and pyridoxal inhibit angiogenesis in serum-free rat aortic ring assay. Int J Mol Med 2001; 8(5):505-508.
62. Loscalzo J. The oxidant stress of hyperhomocyst(e)inemla. J Clin Invest 1996; 98(1):5-7.
63. Misra H P. Generation of superoxide free radical during the autoxidation of thiols. J Biol Chem 1974; 249(7):2151-2155.
64. Harker L A, Slichter S J, Scott C R, Ross R. Homocystinemia. Vascular injury and arterial thrombosis. N Engl J Med 1974; 291(11):537-543.
65. Graham I M, Daly L E, Refsum H M, Robinson K, Brattstrom L E, Ueland P M, Palma-Reis R J, Boers G H, Sheahan R G, Israelsson B, Uiterwaal C S, Meleady R, McMaster D, Verhoef P, Witteman J, Rubba P, Bellet H, Wautrecht J C, de Valk H W, Sales Luis A C, Parrot-Rouland F M, Tan K S, Higgins I, Garcon D, Andria G, et al. Plasma homocysteine as a risk factor for vascular disease. The European Concerted Action Project. JAMA 1997; 277(22):1775-1781.
66. D'Angelo A, Selhub 3. Homocysteine and thrombotic disease. Blood 1997; 90(1): 1-11.
67. Selhub J, Jacques P F, Wilson P W, Rush D, Rosenberg I H. Vitamin status and intake as primary determinants of homocysteinemia in an elderly population. JAMA 1993; 270(22):2693-2698.
68. Brattstrom L, Lindgren A, Israelsson B, Malinow N R, Norrving B, Upson B, Hamfelt A. Hyperhomocysteinaemia in stroke: prevalence, cause, and relationships to type of stroke and stroke risk factors. Eur J Clin Invest 1992; 22(3):214-221.
69. Dalery K, Lusseir-Cacan S, Selhub J, Davignon J, Latour Y, Genest J. Homocysteine and coronary artery disease in French Canadian subjects: relation with vitamins B12, B6, pyridoxal phosphate, and folate. Am J Cardiol. 1995; 75(16):1107-1111.
70. Robinson K, Mayer E L, Miller D P, Green R, van Lente F, Gupta A, Kottke-Marchant K, Savon S R, Selhub J, Nissen S E, et al. Hyperhomocysteinemia and low pyridoxal phosphate. Common and independent reversible risk factors for coronary artery disease. Circulation 1995; 92(10):2825-2830.
71. Clarke R, Collins R. Can dietary supplements with folic acid or vitamin B6 reduce cardiovascular risk? Design of clinical trials to test the homocysteine hypothesis of vascular disease. J Cardiovasc Risk 1998; 5(4):249-255.
72. Omenn G S, Beresford S A, Motulsky A G. Preventing coronary heart disease: B vitamins and homocysteine. Circulation 1998; 97(5):421-424.
73. Boushey C J, Beresford S A, Omenn G S, Motulsky A G. A quantitative assessment of plasma homocysteine as a risk factor for vascular disease. Probable benefits of increasing folic acid intakes. JAMA 1995; 274(13):1049-1057.
74. Homocysteine Lowering Trialists' Collaboration. Lowering blood homocysteine with folic acid based supplements: meta-analysis of randomised trials. BMJ 1998; 316(7135):894-898.
75. Bellamy M F, McDowell I F, Ramsey M W, Brownlee M, Newcombe R G, Lewis M J. Oral folate enhances endothelial function in hyperhomocysteinaemic subjects. Eur J Clin Invest 1999; 29(8):659-662.
76. Woo K S, Chook P, Lolin Y I, Sanderson J E, Metreweli C, Celermajer D S. Folic acid improves arterial endothelial function in adults with hyperhomocystinemia. J Am Coll Cardiol 1999; 34(7):2002-2006.
77. Chambers J C, Ueland P M, Obeid O A, Wrigley J, Refsum H, Kooner J S. Improved vascular endothelial function after oral B vitamins: An effect mediated through reduced concentrations of free plasma homocysteine. Circulation 2000; 102(20): 2479-2483.
78. Vermeulen E G, Stehouwer C D, Twisk J W, van den Berg M, de Jong S C, Mackaay A J, van Campen C M, Visser F C, Jakobs C A, Bulterjis E J, Rauwerda J A. Effect of homocysteine-lowering treatment with folic acid plus vitamin B6 on progression of subclinical atherosclerosis: a randomised, placebo-controlled trial. Lancet 2000; 355(9203):517′-522.
79. Schnyder G, Roffi M, Pin R, Flammer Y, Lange H. Eberli F R, Meier B, Turi Z G, Hess O M. Decreased rate of coronary restenosis after lowering of plasma homocysteine levels. N Engl J Med 2001; 345(22):1593-1600.
80. Vassort G (2001) 81:767-807.
81. Harden T K et al (1994) Annu Rev Pharmacol Toxicol 35:541-579.
82. Dubyak G R et al (1993) Am J Physiol 265:577-606.
83. Di Virigilio et al (2001) Blood 97:587-600.
84. Apasov et al (1995) Immunol Rev 146:5-19.
85. Zoeteweij et al (1996) Hepatology 23:858-865.
86. Zanovello et al (1990) J Immunol 145:1545-1550.
87. Zheng et al (1991) J Cell Biol 112:279-288.
88. Surprenant et al (1996) Science 272:735-738.
89. Wang X et al (1999) Moll Cell Cardiol 31:1063-1072.
90. CanAm Bioresearch (2003) MC1-PCe-08-03-01-360.
91. Trezise D J et al (1994) Eup J Pharm 259:295-300.
92. Lal K J et al (1993) Clin Exper Hypertension 15:489′-500.
93. CanAm Bioresearch (2003) MC1—PCe-02-03-01-47 (not published).
94. Libby P, Ridker P M, Maseri A. Inflammation and atherosclerosis. Circulation 2002; 105(9):1135-1143.
95. Libby P, Harrisson G K. Involvement of the immune system in human atherogenesis: current knowledge and unanswered questions. Lab Invest 1991; 64(1):5-15.
96. Talwar D, Quasim T, McMillan D C, Kinsella J, Williamson C, O'Reilly D S. Pyridoxal phosphate decreases in plasma but not erythrocytes during systemic inflammatory response. Clin Chem 2003; 49(3):515-518.
97. Roubenoff R, Roubenoff R A, Selhub J, Nadeau M R, Cannon J G, Freeman L M, Dinarello C A, Rosenberg I H. Abnormal vitamin B6 status in rheumatoid cachexia. Association with spontaneous tumor necrosis factor alpha production and markers of inflammation. Arthritis Rheum 1995; 38(1):105-109.
98. Chiang E P, Bagley P J, Selhub J, Nadeau M, Roubenoff R. Abnormal vitamin B(6) status is associated with severity of symptoms in patients with rheumatoid arthritis. Am J Med 2003; 114(4):283-287.
99. Kelly P J, Kistler J P, Shih V E, Mandell R, Atassi N, Barron M, Lee H, Silveira S, Furie K L. Inflammation, homocysteine, and vitamin B6 status after ischemic stroke. Stroke 2004; 35(1):12-15.
100. Serfontein W J, Ubbink J B, De Villiers L S, Rapley C H, Becker P J. Plasma pyridoxal-5-phosphate level as risk index for coronary artery disease. Atherosclerosis 1985; 55(3):357-361.
101. Vermaak W J, Barnard H C, Potgieter G M, Marx J D. Plasma pyridoxal-5′-phosphate levels in myocardial infarction. S Afr Med J 1986; 70(4):195-196.
102. Chasan-Taber L, Selhub J, Rosenberg I H, Malinow M R, Terry P, Tishler P V, Willett W, Hennekens C H, Stampfer M J. A prospective study of folate and vitamin B6 and risk of myocardial infarction in US physicians. J Am Coll Nutr 1996, 15(2):136-143.
103. Folsom A R, Nieto F J, McGovern P G, Tsai M Y, Malinow M R, Eckfeldt J H, Hess D L, Davis C E. Prospective study of coronary heart disease incidence in relation to fasting total homocysteine, related genetic polymorphisms, and B vitamins: the Atherosclerosis Risk in Communities (ARIC) study. Circulation 1998; 98(3):204-210.
104. Robinson K, Arheart K, Refsum H, Brattstrom L, Boers G, Ueland P, Rubba P, Palma-Reis R, Meleady R, Daly L, Witteman J, Graham I. Low circulating folate and vitamin B6 concentrations: risk factors for stroke, peripheral vascular disease, and coronary artery disease. European COMAC Group. Circulation 1998; 97(5):437-443.
105. Kelly P J, Shih V, Kistler J P, Barron M, Lee H, Mandell R, Furie K L. Low vitamin B6 but not homocyst(e)ine is associated with increased risk of stroke and transient ischemic attack in the era of folic acid grain fortification. Stroke 2003; 34(6):e51-54.
106. Dinarello C A (1996) Blood 87:2095-2147.
107. Dinarello C A (1998) Int Rev Immunol 16:457-499.
108. Mosely et al (1987) J Biol Chem 262:2941-2944.
109. Thornberry N A et al (1992) Nature 356:768-774.
110. Ceretti D P et al (1992) Science 256:97-100.
111. Rubartelli A et al (1990) EMBO J 9:1503-1510.
112. Hogquist K A et al (1991) J Immunol 147:2181-2186.
113. Perregaux D et al (1992) J Immunol 149:1294-1303.
114. Chin J et al (1993) J Immunol 151:5574-5585.
115. Perregaux D G et al (1998) J Immunol 160:2469-2477.
116. Labiberte R E et al (1999) J Biol Chem 274:36944-36951.
117. Grahames C B A et al (1999) Br J Pharm 127:1915-1921.
118. Perregaux D G et al (1998) Am J Physiol Cell Physiol 275:1538-1547.
119. Labassi J M et al (2002) J Immunol 168:6436-6445.
120. Surprenant A et al (1996) 272-735-738.
121. Perregaux D G et al (1994) J Biol Chem 269:15195-15203.
122. Krishnamurthi S, Westwick J, Kakkar V V. Effect of pyridoxal 5′-phosphate (PALP) on human platelet aggregation, dense granule release and thromboxane B2 generation—role of Schiff base formation. Thromb Haemost 1982; 48(2):136-141.
123. Zahavi M, Zahavi J, Kakkar V V. The effect of pyridoxal-5-phosphate on the inhibition of platelet aggregation and adenosine-3′-5′-cyclic monophosphate accumulation in human platelets. Life Sci 1984; 35(14):1497-1503.
124. van Wyk V, Luus H G, Heyns A D. The in vivo effect in humans of pyridoxal-5′-phosphate on platelet function and blood coagulation. Thromb Res 1992; 66(6):657-668.
125. Palareti G, Salardi S, Piazzi S, Legnani C, Poggi M, Grauso F, Caniato A, Coccheri S, Cacciari E. Blood coagulation changes in homocystinuria: effects of pyridoxine and other specific therapy. J Pediatr 1986; 109(6): 1001-1006.
126. Fonarow, G C. Cleveland Clinic Journal of Medicine, 2003, 70(5), pp. 431-440.
127. Lau et al., Circulation, 2003, 107:32-37.

1. A method of treating a condition selected from the group consisting of vascular inflammation, atherosclerosis, restenosis, plaque destabilization, and bypass graft failure, comprising administering to a patient in need thereof, a therapeutically effective amount of pyridoxal-5′-phosphate or pharmaceutically acceptable salt thereof.

2. The method according to claim 1, wherein the patient is a human.

3. The method according to claim 1, wherein the patient is a diabetic.

4. The method according to claim 1, wherein the therapeutically effective amount of pyridoxal-5′-phosphate is between 0.5 and 50 mg/kg body weight per day.

5. The method according to claim 1, wherein the therapeutically effective amount of pyridoxal-5′-phosphate is between 1 and 15 mg/kg body weight per day.

6. The method according to claim 1, wherein the condition is restenosis and wherein the pyridoxal-5′-phosphate or pharmaceutical salt thereof, is administered by implanting an intravascular stent within an effected artery of the patient wherein the therapeutically effective amount of the pyridoxal-5′-phosphate or pharmaceutically acceptable salt thereof is releaseable from said intravascular stent.

7. The method according to claim 1, further comprising the step of administering a therapeutically effective amount of an anti-inflammatory agent.

8. The method according to claim 1, further comprising the step of administering a therapeutically effective amount of a cardioprotective agent.

9. A method of treating atherosclerosis in a patient suffering thereof comprising administering to the patient a therapeutically effective amount of pyridoxal-5′-phosphate or a pharmaceutically acceptable salt thereof, prior to the patient undergoing a percutaneous coronary intervention.

10. The method according to claim 9, wherein the therapeutically effective amount of the pyridoxal-5′-phosphate or pharmaceutically acceptable salt thereof is administered daily for between 1 and 14 days prior to the percutaneous intervention.

11. The method according to claim 9, wherein the percutaneous coronary intervention is selected from the group consisting of: percutaneous transluminal coronary angioplasty, rotational atherectomy, directional atherectomy, extraction atherectomy, laser angioplasty, implantation of a intracoronary stents and implantation of a catheter.

12. The method according to claim 9, further comprising the step of administering a therapeutically effective amount of pyridoxal-5′-phosphate or a pharmaceutically acceptable salt thereof, following the percutaneous coronary intervention.

13. The method according to claim 12, wherein the therapeutically effective amount of pyridoxal-5′-phosphate or a pharmaceutically acceptable salt thereof is administered daily for between 1 and 30 days following the percutaneous coronary intervention.

14. The method according to claim 12, wherein the therapeutically effective amount of pyridoxal-5′-phosphate or a pharmaceutically acceptable salt thereof is administered daily following the percutaneous coronary intervention for at least 30 days.

15. The method according to claim 9, wherein the therapeutically effective amount of pyridoxal-5′-phosphate is between 0.5 and 50 mg/kg of body weight per day.

16. The method according to claim 9, wherein the therapeutically effective amount of pyridoxal-5′-phosphate is between 1 and 15 mg/kg of body weight per day.

17-20. (canceled)

21. An intravascular stent for use with a narrowed artery wherein said stent has at least one surface reversibly bound with pyridoxal-5′-phosphate or a pharmaceutically acceptable salt thereof.

22. The intravascular stent according to claim 21, wherein at least one surface comprises a physiologically compatible matrix, said matrix adapted for time delayed release of said pyridoxal-5′-phosphate or pharmaceutically acceptable salt thereof, upon implantation of said stent within a patient.

23. The intravascular stent according to claim 21, wherein the amount of the pyridoxal-5′-phosphate or the pharmaceutically acceptable salt thereof is between 10 mg and 10,000 mg.

24. The intravascular stent according to claim 21, wherein the amount of the pyridoxal-5′-phosphate or the pharmaceutically acceptable salt thereof is between 1000 mg and 10,000 mg.

25. The intravascular stent according to claim 21, wherein the amount of the pyridoxal-5′-phosphate or the pharmaceutically acceptable salt thereof is between 1000 mg and 5000 mg.

26. The intravascular stent according to claim 21, wherein the amount of the pyridoxal-5′-phosphate or the pharmaceutically acceptable salt thereof is between 10 mg and 1000 mg.
